(19) United States
(12) Patent Application Publication
(10) Pub. No.: US 2009/0029952 A1
(43) Pub. Date: Jan. 29, 2009

(54) UNIQUE COMBINATION OF ACETYLSALICYLIC ACID, ETHYLENEDIAMINE DIHYDROIODIDE (EDDI), AND BUFFER FOR USE IN ANIMAL DRINKING WATER

(76) Inventors: Bryan Page, Gardner, NC (US); Stephen F. Driggers, Marietta, GA (US); David L. Wilson, Commerce, GA (US)

Correspondence Address: E. J. Asbury III, Esq.;E. J. Asbury III, LLC, 1265 Lake Colony Dr., Marietta, GA, 30068, US

(21) Appl. No.: 12/062,503

(22) Filed: Apr. 3, 2008

Related U.S. Application Data
(60) Provisional application No. 60/909,771, filed on Apr. 3, 2007.
(60) Provisional application No. 60/912,615, filed on Apr. 18, 2007.

(51) Int. Cl. A61K 31/60

(52) U.S. Cl. 514/162

(57) ABSTRACT

A unique combinations include acetylsalicylic acid (aspirin), ethylenediamine dihydriodide (EDDI), potassium iodide, sodium acetate, and sodium diacetate, useful in helping poultry, swine, and cattle overcome certain symptoms after vaccination. The inventive combinations readily dissolve in water at room temperature to form an effective concentration for inclusion in the animal's drinking water.

### CROSS-REFERENCE TO RELATED APPLICATION

This application claims the benefit of U.S. Provisional Application Ser. No. 60/909,771, filed on Apr. 3, 2007, and claims the benefit of U.S. Provisional Application Ser. No. 60/912,615, filed on Apr. 18, 2007, and the entirety of each is hereby incorporated herein by reference.

### BACKGROUND OF THE INVENTION

1. Field of the Invention

The present invention generally relates to compounds and processes used in animal nutrition. More particularly, the present invention relates to a useful composition for helping cattle, swine, and poultry overcome certain symptoms after illness or vaccination, and which composition is readily water soluble.

2. Description of the Related Art

Livestock such as poultry, swine and cattle are typically supplied drinking water via an automatic low pressure watering system. The animal watering system, sometimes refereed to as a dripper system, provides a constant slow source of hydration for the animals. Nutritional supplements which are water soluble may be added to the drinking water within the automatic watering system. The watering systems commonly employ an injection system for injecting liquid supplement into the drinking water at a known concentration and rate. The watering system, and integral supplement injection system commonly have small orifices to control the water flow rate. Any particulate matter within the drinking water or the injected supplement will easily clog the low pressure orifices within the low pressure animal watering system. When the orifices are clogged, animals are denied both basic drinking water, and the desired nutritional supplement.

Any interruption in the drinking water supply to commercially grown livestock is undesirable. If the livestock is denied water for any period, or the animal reduces normal water intake due to stress from vaccination, the animal health suffers. The animal weight gain during that portion of the growth cycle is impaired, and the total animal weight at the completion of the growth cycle is reduced. Commercial livestock operations have a limited number of days for each animal to achieve full weight prior to market. As livestock are sold in commercial markets by weight, this results in a measurable loss in profit to the livestock farmer.

Many bacterial and viral diseases can stress livestock during the growth cycle. Poultry are subject to a range of diseases which require some form of vaccination. A recent development is that certain respiratory diseases that were once a sporadic problem are now widespread over large geographical areas. Common practice is to vaccinate entire flocks in anticipation of outbreaks. Vaccination can be done at the hatchery or more communally at about 10 days of age when chick are the confined in the growing facility. Among diseases commonly vaccinated for in the growing facilities are: Infectious Laryngotracheitis, New Castle Disease, Reo Virus, Merk's Disease and Infectious Bursal Disease. When animals are vaccinated for these diseases mild symptoms of the diseases occur. For reparatory diseases these may include fever, lack of appetite, general sluggishness, “huddling”, reduced water intake, cough, watery eyes, sneezing, and increased mortality. As with poultry, both swine and cattle use similar vaccine regimes. After vaccination, both swine and cattle producing flu like symptoms manifesting as fever, respiratory distress, depression, lack of appetite, and increased mortality.

Acetylsalicylic acid (aspirin) is well recognized as an analgesic to relieve muscle and joint aches and as an aid to reduce fevers in cattle, swine, and poultry. Unfortunately, aspirin has a low solubility in water. Even in low concentrations, acetylsalicylic acid will tend to float on the top, or settle to the bottom of the container. Further addition of acetylsalicylic acid to the solution will result in a particulate substrate settling within the solution.

Iodine is an important nutritional supplement for the animal, especially during the stress of sickness or vaccination. Potassium iodide is useful as a nutrition source of iodine for cattle, swine, and, poultry. It is usually marketed in combination with dextrose or sodium chloride at 4.0%-10.0% inclusion. It is also marketed an ingredient in multi-vitamin mixtures for livestock of livestock feeds. Potassium iodine is soluble in water, however, is not normally soluble with aspirin at inclusion levels commonly accepted as effective for the treatment of the post vaccination symptoms.

Ethylenediamine dihydriodide (“EDDI”) or organic iodine has the same properties of potassium iodine. One of the advantages is that it is organic in nature. However, it is also not generally soluble with aspirin. EDDI is useful as a nutrition source of iodine for cattle, swine, and, poultry. It is usually marketed in combination with dextrose or sodium chloride at 4.6%-9.2% inclusion. It is also marketed as an ingredient in multi-vitamin mixtures for livestock of livestock feeds.

Elemental iodine is much more commonly available form of iodine. However, elemental iodine is not soluble in water. It is sold as an iodophor, which is iodine in a surfactant—phosphoric acid—water solution. It is used in drinking water as a disinfectant. However, this product is not compatible with aspirin and or caffeine due to the chemical nature of the iodophor. Elemental Iodine in the form of an iodophor will mix with water. However, a given volume of feed water at room temperature, is only capable of absorbing so many ions in solution. Under normal conditions, a mixture of elemental iodine and aspirin will readily have particulate solids settling within the solution, or floating on the surface.

Compatibility problems arise when mixing more than a limited number of the desired components together. For example, attempts to mix iodophor with aspirin and caffeine in a volume of water commonly encountered in the metering systems that add solutions to livestock drinking water lines fail due to solubility problems. Particulate matter of aspirin will float to the top of the container, and possibly iodine will settle to the bottom of the container as a substrate within the solution. Any particulate matter will quickly clog the orifices within the system.

Since both Aspirin and iodine may not be present in the low pressure water system at the same time in effective concentrations due to solubility problems, a common approach in the art has been to add an aspirin or aspirin/caffeine treatment to drinking water for a period of time, followed by a cleaning of the water line, followed by the addition of an iodophor solution. This is undesirable in a livestock production environment in that the animal is receiving one, or the other, of the desired nutritional supplements aspirin and iodine, but not both in effective quantities at the same time. The additional task of cleaning the system and keeping track of the supplement additions is also problematic.

Accordingly, what is needed in the art is a composition which may provide a combination of desired ingredients which exhibits the ability to mitigate multiple symptoms and side effects from the vaccination program. The composition should provide both acetylsalicylic acid and an iodine source in concentrations effective for the animal treatment. The composition should be readily water soluble at room temperature, and should be water soluble at elevated concentrations for use in the injection systems of animal drinking water system. It is to such a composition that the present invention is directed.

### SUMMARY OF THE INVENTION

The disadvantages of the prior art are overcome by the present invention which, in one aspect, is a composition for the treatment of livestock, the composition being readily dissolved in water, the composition including acetylsalicylic acid in a concentration between 12%-20% by weight, ethylenediamine dihydroiodide (EDDI) in a concentration between 5%-18% by weight, and a buffer in a concentration between 40%-83% by weight. The buffer may be sodium acetate or sodium diacetate, or a combination thereof. The livestock to be treated ty the composition may be poultry, swine, or cattle. The composition may be water soluble at concentrations of up to 12% by weight.

In another aspect, the composition for the treatment of poultry may be acetylsalicylic acid in a concentration between 12%-20% by weight; ethylenediamine dihydroiodide (EDDI) in a concentration between 5%-9% by weight; and a buffer in a concentration between 40%-83% by weight.

In yet another aspect, the composition for the treatment of swine may be acetylsalicylic acid in a concentration between 12%-20% by weight; ethylenediamine dihydroiodide (EDDI) in a concentration between 10%-18% by weight; and a buffer in a concentration between 40%-78% by weight.

In yet another aspect, the invention presents a composition for the treatment of livestock, the composition being readily dissolved in water, the composition including acetylsalicylic acid in a concentration between 12%-20% by weight, potassium iodide in a concentration between 3%-6% by weight, and a buffer in a concentration between 40%-85% by weight. The buffer may be sodium acetate or sodium diacetate, or a combination thereof. The composition is water soluble at inclusion rates up 12 percent by weight.

In yet another aspect, the invention presents a process for the treatment of livestock, the livestock being treated by ingestion of a composition, including acetylsalicylic acid in a concentration between 12%-20% by weight, ethylenediamine dihydroiodide (EDDI) in a concentration between 5%-18% by weight, a buffer in a concentration between 40%-83% by weight; and wherein the composition is ingested via dissolving the composition within the livestock drinking water at an effective concentration. The buffer may be sodium acetate or sodium diacetate, or a combination thereof. The livestock may be poultry, swine, or cattle, and the effective concentration of the composition within the animal drinking water is at least 2 parts per million.

The composition can be pre-mixed with water within an injection system tank to form a pre-mix solution, and the pre-mix solution is then injected into the livestock drinking water to form the effective concentration. The concentration of the composition within the pre-mix solution in the injection system tank may be between 0.3 to 12 percent by weight.

In yet another aspect of the present invention, a composition for the treatment of livestock may be acetylsalicylic acid in a concentration between 12%-20% by weight, ethylenediamine dihydroiodide (EDDI) in a concentration between 5%-9% by weight, a buffer in a concentration between 40%-83% by weight; and the effective concentration is at least 2 parts per million in the poultry drinking water.

In yet another aspect of the present invention, a composition for the treatment of swine may be acetylsalicylic acid in a concentration between 12%-20% by weight, ethylenediamine dihydroiodide (EDDI) in a concentration between 10%-18% by weight, a buffer in a concentration between 40%-78% by weight; and the effective concentration is at least 2 parts per million in the swine drinking water.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE DRAWINGS

FIG. 1 is the average chick body temperature over test period.

FIG. 2 is a plot of the average body weight over test period.

FIG. 3 is a plot of the ambient temperature of poultry house over the test period.

FIG. 4 is a plot of the daily water consumption of the flock over the test period.

FIG. 5 is a plot of the cumulative mortality of the flock over the test period.

### DETAILED DESCRIPTION OF THE INVENTION

The invention presents a unique combination of desired ingredients which exhibits the ability to mitigate multiple symptoms and side effects from the vaccination program. The unique combinations include acetylsalicylic acid (aspirin), ethylenediamine dihydriodide (EDDI), potassium iodide, sodium acetate, sodium diacetate, and caffeine are useful in helping poultry, swine, and cattle overcome certain symptoms after vaccination. In poultry, this would include the absence of fever, huddled chicks, sneezing, coughing, mucosal discharge, water and feed intake reduction, and mortality. The inventive combinations readily dissolve in water at room temperature to form an effective concentration for inclusion in the animal's drinking water.

Sodium Acetate and sodium diacetate are buffers and sources of acetate ion which increases thirst in poultry, thereby, increasing desirable water consumption. Sodium acetate and sodium diacetate also have the property of helping to increase the solubility compatibility of aspirin when in the presence of sources of iodine in water. With the additional of sodium acetate and sodium diacetate the solubility of acetylsalicylic acid in the presence of iodine in water at room temperature may be increased to a maximum of approximately 50% by weight of the composition of the formula. As will be appreciated by those skilled in the art, other buffers may be used to include, but not limited too, monosodium phosphate, disodium phosphate, sodium hydrogen sulfate, the potassium or sodium salts of propionate, fumaric, lactic, malic, maleic, and formic acids.

The buffered organic acid composition of the present invention releases acetic acid upon dissolving in water at a level that optimizes the pH of the system. Animals increase water uptake at slightly acidic pH levels. While EDDI is soluble in water, aspirin is not. It is known in the art that acetylsalicylic acid is chemically more soluble in the presence of acetates and at slightly acidic pH's in the range of 5-7. In the present invention, the acetates make the combination of acetylsalicylic acid and organic iodine more compatible than what would be expected from their individual solubility data. From chemical handbooks, aspirin has solubility in water of 0.1%. The inventive composition has the ability to deliver an aspirin solution of up to 2.5% resulting in a much more effective concentration within the animal drinking water.

In a first embodiment, the present invention incorporates a unique formulation which allows addition of all the discussed desired components at therapeutic levels to animal drinking water via in-line injection from a stock concentration solution. The inclusion rates by weight are acetylsalicylic acid (aspirin) 1% to 50%, sodium acetate 1% to 90%, sodium diacetate 1% to 90%, ethylenediamine dihydriodide (EDDI) 0% to 50%, potassium iodide 0% to 50%, caffeine 0-5%. This combination is a stable; water soluble mixture that can be added to livestock drinking water for relief of symptoms of sluggishness, and fever and respiratory problems after administering certain vaccinations and is a good source of nutrient iodine during the stressful period after vaccination. The formulation is effective in poultry, including chickens, turkeys, ducks, geese, and other production fowl, swine, and cattle production. The appropriate dosage is composition concentrations within the range of 2 to 6 parts per million in the animal drinking water.

In another embodiment of the present invention, a mixture formulation effective in the production of poultry is; 12%-20% acetylsalicylic acid, 5%-9% ethylenediamine dihydriodide (EDDI), ½% strawberry flavoring, and the balance of the composition as sodium acetate. The strawberry flavoring is commercially offered under the brand BFI Strawberry Aid.

In an alternative embodiment of the present invention, another formulation effective in the production of poultry is; 12%-20% acetylsalicylic acid, 3% to 6% potassium iodide, ½% strawberry flavoring, and the balance of the composition as sodium acetate.

In another alternative embodiment, a more specific formulation for use in the production of poultry is 16% acetylsalicylic acid, 7% ethylenediamine dihydriodide (EDDI), ½% strawberry flavoring, and the balance of the composition as sodium acetate.

In another embodiment of the present invention, a mixture formulation effective in the production of swine is; 12%-20% acetylsalicylic acid, 10% to 18% ethylenediamine dihydroiodide (EDDI), ½% strawberry flavoring, and the balance of the composition as sodium acetate

In an alternative embodiment of the present invention, another formulation effective in the production of swine is 12%-20% acetylsalicylic acid, 3% to 6% potassium iodide, ½% strawberry flavoring, and the balance of the composition as sodium acetate.

In another alternative embodiment, a more specific formulation for use in the production of swine is; 16% acetylsalicylic acid, 14% ethylenediamine dihydroiodide (EDDI), ½% strawberry flavoring, and the balance of the composition as sodium acetate

As previously stated, the solubility of aspirin in the composition is greatly improved over the solubility of aspirin in water. For example, the maximum solubility of an example composition of 16% acetylsalicylic acid, 7% ethylenediamine dihydriodide (EDDI), ½% strawberry flavoring, and a balance of sodium acetate can reach 12 percent by weight. In the resulting mixture, the concentration of aspirin will be 2.5%, and the concentration of EDDI will 0.08%. By comparison, the maximum solubility of aspirin alone in water, or in the presence of sodium acetate or sodium diacetate is only 0.1% by weight.

The common means of delivering the formulation to the livestock of interest is inclusion in the drinking water system at an appropriate mixture rate. The composition may be added to the injection system tank at concentrations up to the maximum solubility of 12 percent by weight. To deliver an effective dosage to the animal via the injection system, the concentration in the injection system tank should be at least 0.3% by weight. For applications in poultry production, a mixture rate of 280 grams of the formulation to 640 gallons of the poultry drinking water is effective. This results in approximately a 4 parts per million concentration of the composition in the final drinking water. Composition concentrations within the range of 2 to 6 parts per million in the animal drinking water are effective.

### EXAMPLE 1

Infectious Laryngotracheitis is a herpes virus of variable pathogenicity effecting poultry. Transmission is rapid and infected birds remain carriers. Transmission is easily done through direct exposure to respiratory aerosols and contaminated feed, water, personnel, equipment, and other factors.

The start of disease is associated with conjunctivitis, lacrimation, and nasal discharge, followed by coughing, sneezing, rates. Some birds show gasping, depression and lack of appetite. A bloody mucous may be expelled. Morbidity is usually up to 90% and mortality may be from 2-70%. Recovery usually takes 3 to 5 weeks.

The common practice in the poultry industry is to vaccinate chicks in areas that have experienced outbreaks of disease. Besides the cost of vaccination, there are well recognized problems associated with vaccination of chicks for Infectious Laryngotracheitis. Vaccination usually results in flu like symptoms on or after the fourth day continuing for up to 10 days after on set. These symptoms are sluggishness, fever (on the order of 2° F. elevated temperature), and respiratory distress (manifesting as nasal discharge, coughing and sneezing). Normal healthy chick temperature at age 15 to 25 days is approximately 107° F. These symptoms further manifest themselves as reduced feed and water intake compared to normal healthy chicks. Subsequently, the bird weight during the remainder of the grow-out suffers as compared to that of healthy birds.

A composition of 16% acetylsalicylic acid, 7% ethylenediamine dihydroiodide (EDDI), ½% strawberry flavoring, and the balance sodium acetate was administered to the poultry via the watering system. The composition was dissolved within the animal drinking system's injector tank. The concentrated pre-mix was then injected into the animal drinking water.

The composition was administered at the fourth day after vaccination and before symptoms of fever, flu like systems and respiratory symptoms appeared. Treatment protocol is 1 ea 280 gm packet of the composition dissolved into five gallons of water within the injection system tank of the watering system. The resulting pre-mix solution was metered at 1 oz to 128 oz of drinking water, or approximately a 4 ppm concentration in the animal drinking water. The composition was added to the drinking water for a period 10 days.

The normal consequence of vaccination of 10-15 day old chicks for Infectionous Lamgotracheitis is an incubation period of 4 to 10 days post vaccination. This period is generally characterized by normal or near normal growth. The first noticeable symptoms are generally lacrimation, nasal discharge, sneezing, and rales. Followed within 24-48 hours fever up to 2° F. and noticeably decreased activity (running and chirping). Feed uptake and water uptake decrease, leading to reduced weight gain. Lastly, a spike in chick mortality is usually observed. It is also not uncommon to observe what is known as a “rolling outbreak” (all the chicks do not show symptoms immediately; every 7 to 10 days there are new groups of chicks showing symptoms).

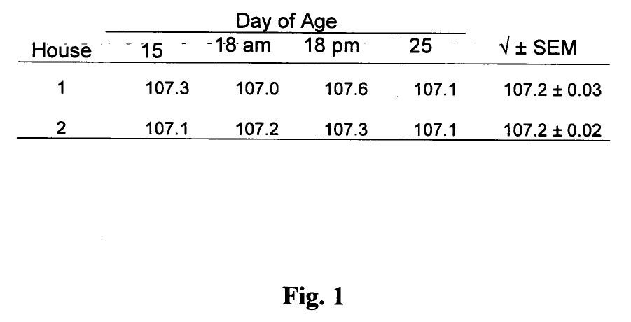

FIG. 1 presents the average chick body temperature over the test period. The test data demonstrates no increase in average chick body temperature. The temperatures of chicks were taken rectally. The consistent temperatures during the treatment indicate no increase in body temperature due to fevers which would normally be expected. A rise of 2 degrees F. is expected in chicks. The temperatures remained normal for the life of the treatment and the life of the flock. The normal temperature profile for the life of the flock indicates the absence of “rolling outbreak”.

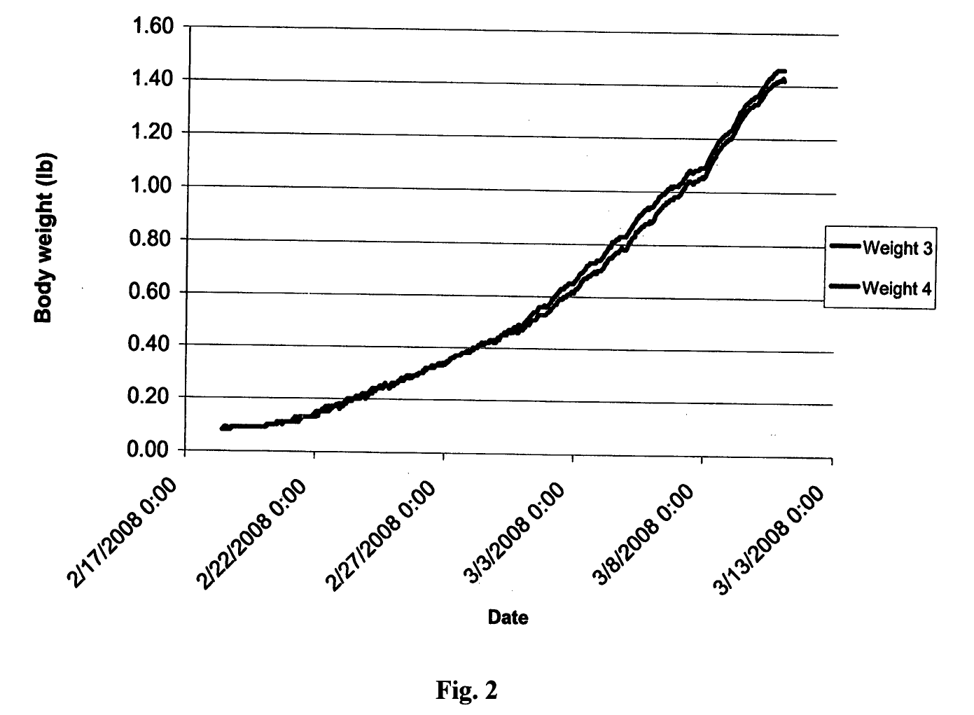

FIG. 2 presents the average body weight gain over the test period. Each house has four sets of scales. Random body weights are automatically taken every hour. The graph shows no decrease the rate of body weight gain. Under normal circumstances vaccinated chicks show a decrease in the rate of weight gain between the 4 and 10th days past vaccination. This is due to a lack of feed and water intake.

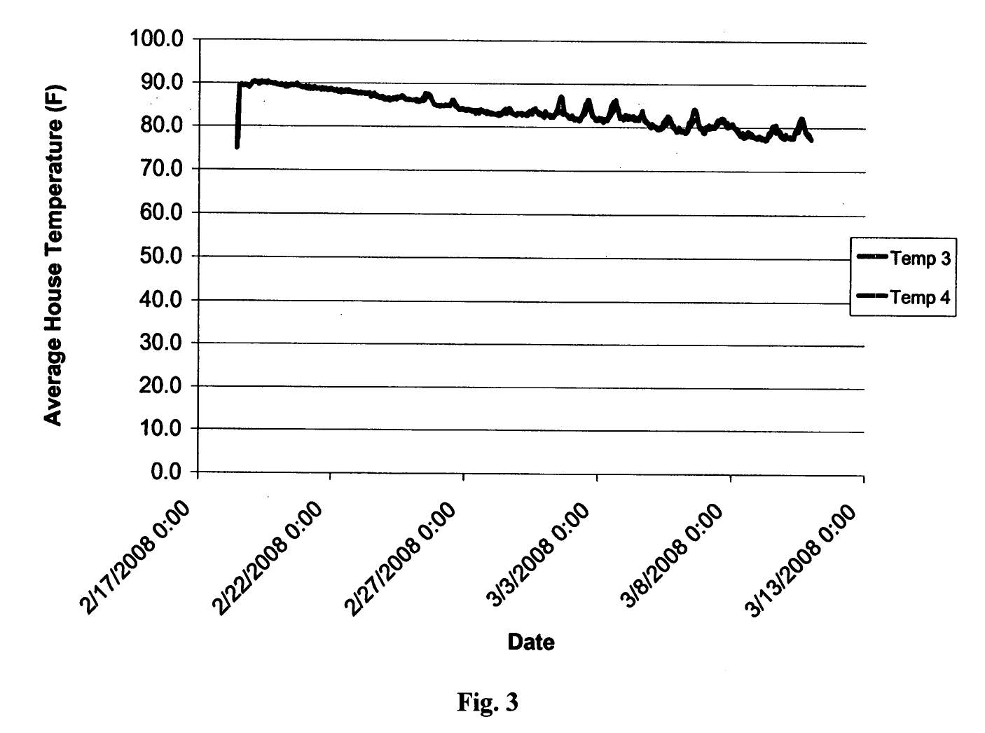

FIG. 3 demonstrates that the poultry house temperature is in the planned temperature profile. The house temperatures are dropped as the birds age. The 2 degree F. body temperature increase that is usually experienced takes into account this normal temperature profile. This flock was raised under normal house temperature profiles.

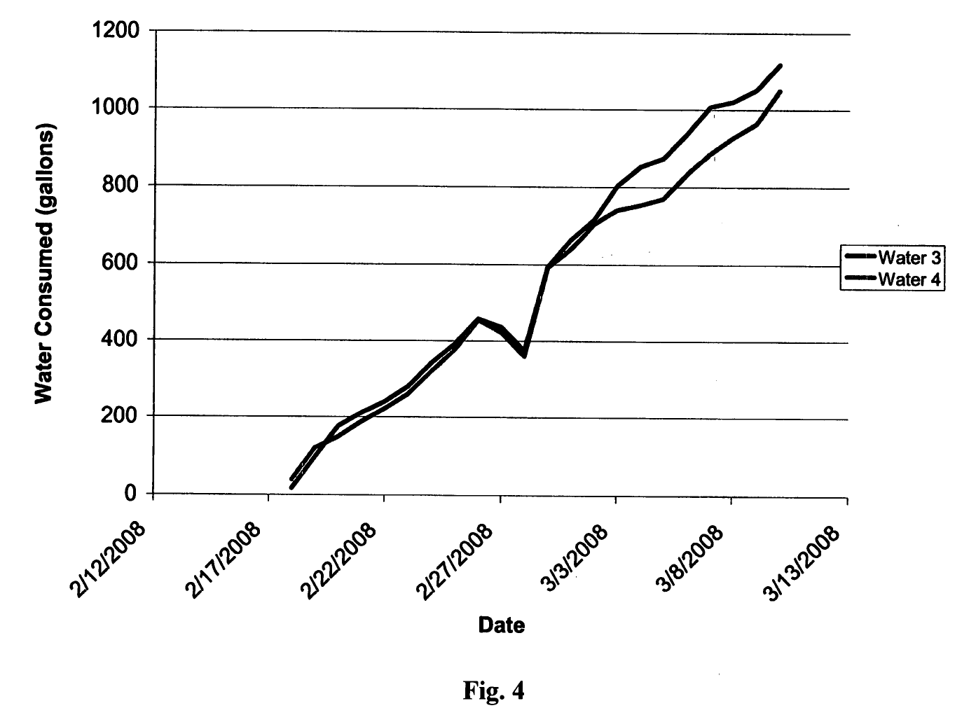

FIG. 4 depicts the daily water consumption of the test flock. It is normally expected for water consumption to decrease 20-25% at the onset of symptoms post vaccination. The flock was vaccinated on Feb. 28, 2008. A drop in water consumption can be observed on that day. This drop was due to the normal removal of water supply for vaccination purposes. As soon as water supply was resumed, water consumption return to the normal profile for healthy birds. At 48 hours post vaccination a drop of 20-25% in water consumption is expected to be observed. No such drop in water consumption was observed.

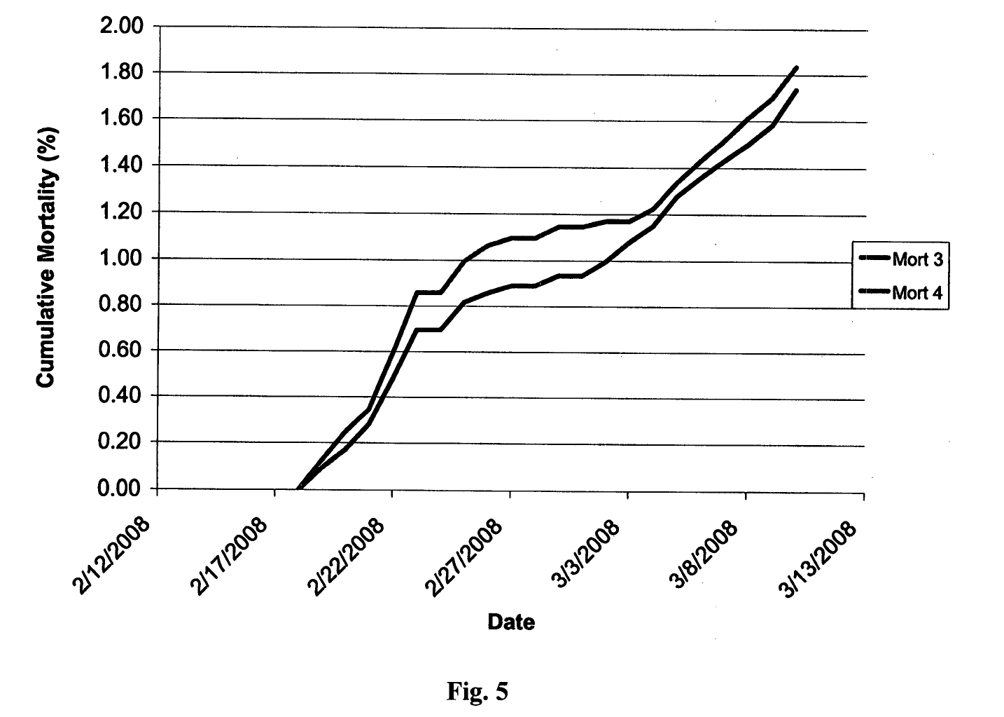

FIG. 5 depicts the cumulative chick mortality in the test flock. At 48 hours post vaccination up to 10 days post vaccination an increase in mortality of up to 4% may be expected. An increase number of purposefully culled birds were noticed in house 3. However, the producer noted that that house experienced higher lever of ammonia, an unrelated factor to vaccination, which resulted in blindness of the chick. This producer destroys those animals that are blind as a good management practice. No increase in mortally was noted that could bet attributed to post vaccination symptoms or complications.

As can be seen from the foregoing test data, none of the typical adverse symptoms expected in the vaccinated chicks were observed when the inventive composition was supplied via the poultry drinking water. The birds exhibit a normal temperature, steady body weight gain, steady water consumption, and an average mortality.

All of the compositions and process disclosed and claimed herein can be made and executed without undue experimentation in light of the present disclosure. While the compositions and process of this invention have been described in terms of preferred embodiments, it will be apparent to those of skill in the art that variations may be applied to the compositions and process and in the steps, or in the sequence of steps, of the methods described herein without departing from the concept, spirit, and scope of the invention. More specifically, it will be apparent that certain chemical compounds which are both chemically related, and similar physical characteristics, may be substituted for the chemical compounds described herein while the same or similar results would be achieved. All such similar substitutes and modifications apparent to those skilled in the art are deemed to be within the spirit, scope, and concept of the invention.

We claim:

1. A composition for the treatment of livestock, the composition comprising;
acetylsalicylic acid in a concentration between 12%-20% by weight;
ethylenediamine dihydroiodide (EDDI) in a concentration between 5%-18% by weight;
a buffer in a concentration between 40%-83% by weight; and
the composition being readily water soluble.

2. The composition of claim 1, wherein the buffer comprises at least one of sodium acetate and sodium diacetate.

3. The composition of claim 1, wherein the livestock to be treated comprises poultry.

4. The composition of claim 1, wherein the livestock to be treated comprises swine.

5. The composition of claim 1, wherein the livestock to be treated comprises cattle.

6. The composition of claim 1, further comprising;
acetylsalicylic acid in a concentration between 12%-20% by weight;
ethylenediamine dihydroiodide (EDDI) in a concentration between 5%-9% by weight;
a buffer in a concentration between 40%-83% by weight; and
wherein the composition is for the treatment of poultry.

7. The composition of claim 1, further comprising;
acetylsalicylic acid in a concentration between 12%-20% by weight;
ethylenediamine dihydroiodide (EDDI) in a concentration between 10%-18% by weight;
a buffer in a concentration between 40%-78% by weight; and
wherein the composition is for the treatment of swine.

8. The composition of claim 1, wherein the composition is water soluble at inclusion rates of up to 12 percent by weight.

9. A composition for the treatment of livestock, the composition comprising;
acetylsalicylic acid in a concentration between 12%-20% by weight;
potassium iodide in a concentration between 3%-6% by weight;
a buffer in a concentration between 40%-85% by weight; and
the composition being readily water soluble.

10. The composition of claim 9, wherein the buffer comprises at least one of sodium acetate and sodium diacetate.

11. The composition of claim 9, wherein the composition is water soluble at inclusion rates up 12 percent by weight.

12. A process for the treatment of livestock, the livestock being treated by ingestion of a composition, the composition comprising;
acetylsalicylic acid in a concentration between 12%-20% by weight;
ethylenediamine dihydroiodide (EDDI) in a concentration between 5%-18% by weight; and
a buffer in a concentration between 40%-83% by weight; and
wherein the composition is ingested via dissolving the composition within the livestock drinking water at an effective concentration.

13. The process of claim 12, wherein the buffer comprises at least one of sodium acetate, and sodium diacetate.

14. The process of claim 12, wherein the livestock are poultry, and the effective concentration of the composition within the poultry drinking water is at least 2 parts per million.

15. The process of claim 12, wherein the livestock are swine, and the effective concentration of the composition within the swine drinking water is at least 2 parts per million.

16. The process of claim 12, wherein the livestock are cattle, and the effective concentration of the composition within the cattle drinking water is at least at least 2 parts per million.

17. The process of claim 12, wherein the composition is pre-mixed with water within an injection system tank to form a pre-mix solution, and the pre-mix solution is injected into the livestock drinking water to form the effective concentration.

18. The process of claim 17, wherein the concentration of the composition within the pre-mix solution in the injection system tank is between 0.3 to 12 percent by weight.

19. The process of claim 12, further comprising;
acetylsalicylic acid in a concentration between 12%-20% by weight;
ethylenediamine dihydroiodide (EDDI) in a concentration between 5%-9% by weight;
a buffer in a concentration between 40%-83% by weight; and
wherein the process is for the treatment of poultry, and the effective concentration is at least 2 parts per million in the poultry drinking water.

20. The process of claim 12, further comprising;
acetylsalicylic acid in a concentration between 12%-20% by weight;
ethylenediamine dihydroiodide (EDDI) in a concentration between 10%-18% by weight;
a buffer in a concentration between 40%-78% by weight; and
wherein the process is for the treatment of swine, and the effective concentration is at least 2 parts per million in the swine drinking water.
